(19) United States
(12) Patent Application Publication
(10) Pub. No.: US 2020/0405334 A1
(43) Pub. Date: Dec. 31, 2020

(54) TISSUE SPECIMEN RETRIEVAL DEVICES

(71) Applicant: COVIDIEN LP, MANSFIELD, MA (US)

(72) Inventors: JACOB C. BARIL, NORWALK, CT (US); SAUMYA BANERJEE, HAMDEN, CT (US)

(21) Appl. No.: 16/455,844

(22) Filed: Jun. 28, 2019

(51) Int. Cl. A61B 17/221; A61B 17/00; A61B 10/02; A61B 10/00

(52) U.S. Cl. CPC A61B 17/221; A61B 17/00234; A61B 17/50; A61B 10/0096; A61B 2017/00287; A61B 10/02

(57) ABSTRACT

A tissue specimen retrieval device includes a first shaft and a second shaft telescopically movable relative to the first shaft. The second shaft supports an end effector assembly. The second shaft is movable relative to the first shaft between a retracted position and a deployed position. The end effector assembly includes a tissue specimen bag supported by a first arm and a second arm. The first and second arms open the tissue specimen bag when the second shaft is in the deployed position. The tissue specimen bag includes an insertion cap disposed on and extending outwardly from an exterior surface of the tissue specimen bag. The insertion cap is operably engaged with the distal portion of the first shaft when the second shaft is in the retracted position to define an atraumatic insertion tip.

### BACKGROUND

### Technical Field

The present disclosure relates to tissue specimen retrieval from an internal body cavity and, more particularly, to tissue specimen retrieval devices to facilitate retrieval of a tissue specimen from an internal body cavity.

### Background of Related Art

In minimally-invasive surgical procedures, operations are carried out within an internal body cavity through small entrance openings in the body. The entrance openings may be natural passageways of the body or may be surgically created, for example, by making a small incision into which an access device is inserted.

Minimally-invasive surgical procedures may be used for partial or total retrieval of a tissue specimen from an internal body cavity. However, the restricted access provided by minimally-invasive openings (natural passageways and/or surgically created openings) presents challenges with respect to maneuverability and visualization. The restricted access also presents challenges when the tissue specimen is required to be removed. As such, a tissue specimen that is deemed too large for intact retrieval may be broken down into a plurality of smaller pieces to facilitate retrieval from the internal body cavity.

During such minimally-invasive surgical procedures, it is common that a cyst, tumor, or other affected tissue specimen is required to be removed. In these and other procedures where cancerous tissue is required to be removed, retrieval of the tissue specimen in an enclosed environment is highly desirable to inhibit seeding of cancer cells. Thus, with respect to breaking down large tissue specimens for retrieval through minimally-invasive openings, there is the added challenge of doing so within an enclosed environment.

### SUMMARY

In accordance with an aspect of the present disclosure, a tissue specimen retrieval device includes a first shaft and a second shaft telescopically movable relative to the first shaft. The second shaft supports an end effector assembly at a distal end portion thereof. The second shaft is movable relative to the first shaft between a retracted position, in which the end effector assembly is disposed within the first shaft, and a deployed position, in which the end effector assembly extends distally from the first shaft. The end effector assembly includes a tissue specimen bag supported by a first arm and a second arm. The first and second arms open the tissue specimen bag when the second shaft is in the deployed position. The tissue specimen bag includes an insertion cap disposed on and extending outwardly from an exterior surface of the tissue specimen bag. The insertion cap is operably engaged with the distal portion of the first shaft when the second shaft is in the retracted position to define an atraumatic insertion tip.

In some aspects, the insertion cap includes a proximal portion configured to be inserted into a distal portion of the first shaft when the second shaft is in the retracted position. The insertion cap also includes a rounded distal portion.

In some aspects, an outer circumference of the rounded distal portion of the insertion cap is substantially aligned with an outer circumference of the distal portion of the first shaft when the second shaft is in the retracted position.

In some aspects, the insertion cap is operably engaged with the tissue specimen bag.

In some aspects, the insertion cap is positioned at a bottom portion of the tissue specimen bag.

In some aspects, the tissue specimen bag is disposed within the first shaft when the second shaft is in the retracted position.

In some aspects, a first handle is disposed at a proximal portion of the first shaft and a second handle is disposed at a proximal portion of the second handle. The first and second handles are relatively movable to move the second shaft between the retracted and deployed positions. The first handle includes first and second finger loops.

In some aspects, the proximal portion of the insertion cap has a narrower width than a width of the rounded distal portion of the insertion cap.

In some aspects, the insertion cap is locatable to facilitate location of a bottom portion of the tissue specimen bag.

In some aspects, the insertion cap includes or is formed of plastic or silicon.

In some aspects, the first and second arms of the end effector assembly are configured to close the tissue specimen bag when the second shaft is in the retracted position.

In some aspects, the first and second arms of the end effector assembly are configured to deploy the insertion cap and the tissue specimen bag from the first shaft when the second shaft is in the deployed position.

In accordance with an aspect of the present disclosure, a tissue specimen retrieval device includes a first shaft and a second shaft telescopically movable relative to the first shaft. The second shaft supports an end effector assembly at a distal end portion thereof. The second shaft is movable relative to the first shaft between a retracted position, in which the end effector assembly is disposed within the first shaft, and a deployed position, in which the end effector assembly extends distally from the first shaft. The end effector assembly includes a tissue specimen bag supported by a first arm and a second arm. The first arm includes a first hinge and the second arm includes a second hinge. The first and second arms open the tissue specimen bag about the first and second hinges, respectively, when the second shaft is in the deployed position. The tissue specimen bag includes an insertion cap disposed on and extending outwardly from an exterior surface of the tissue specimen bag. The insertion cap is operably engaged with the distal portion of the first shaft when the second shaft is in the retracted position to define an atraumatic insertion tip.

### BRIEF DESCRIPTION OF THE DRAWINGS

The accompanying drawings, which are incorporated in and constitute a part of this specification, illustrate embodiments of the present disclosure and, together with the detailed description below, serve to further explain the present disclosure, in which:

FIG. 1 is a perspective view of a tissue specimen retrieval device provided in accordance with aspects of the present disclosure, disposed in a retracted position;

FIG. 2 is a perspective view of the tissue specimen retrieval device of FIG. 1, disposed in a deployed position;

FIG. 3 is an enlarged, perspective view of the end effector assembly of the tissue specimen retrieval device of FIG. 1 with the specimen bag removed therefrom;

FIGS. 4A and 4B are side views of the end effector assembly of the tissue specimen retrieval device of FIG. 1 illustrating a first collapsing of the end effector assembly;

FIGS. 5A and 5B are top views of the end effector assembly of the tissue specimen retrieval device of FIG. 1 illustrating a second collapsing of the end effector assembly;

FIG. 6A is a perspective view of a tissue specimen retrieval device including an insertion cap disposed in a retracted position in accordance with aspects of the present disclosure;

FIG. 6B is an enlarged perspective view of a distal end of a tissue specimen retrieval device including an insertion cap disposed in a retracted position in accordance with aspects of the present disclosure;

FIG. 7 is a perspective view of the tissue specimen retrieval device of FIGS. 6A and 6B disposed in a deployed position;

FIG. 8A is an enlarged perspective view of the insertion cap of FIGS. 6A and 6B disposed in a deployed position; and

FIG. 8B is an enlarged perspective view of the insertion cap of FIGS. 6A and 6B disposed in a retracted position.

### DETAILED DESCRIPTION

As used herein, the term “distal” refers to the portion that is being described which is further from a user, while the term “proximal” refers to the portion that is being described which is closer to a user. Further, to the extent consistent, any of the aspects and features detailed herein may be used in conjunction with any or all of the other aspects and features detailed herein.

As used herein, the terms parallel and perpendicular are understood to include relative configurations that are substantially parallel and substantially perpendicular up to about + or −10 degrees from true parallel and true perpendicular.

“About” or “approximately” as used herein may be inclusive of the stated value and means within an acceptable range of variation for the particular value as determined by one of ordinary skill in the art, considering the measurement in question and the error associated with measurement of the particular quantity (e.g., the limitations of the measurement system). For example, “about” may mean within one or more standard variations, or within ±30%, 20%, 10%, 5% of the stated value.

Descriptions of technical features or aspects of an exemplary embodiment of the present disclosure should typically be considered as available and applicable to other similar features or aspects in another exemplary embodiment of the present disclosure. Accordingly, technical features described herein according to one exemplary embodiment of the present disclosure may be applicable to other exemplary embodiments of the present disclosure, and thus duplicative descriptions may be omitted herein.

Exemplary embodiments of the present disclosure will be described more fully below (e.g., with reference to the accompanying drawings). Like reference numerals may refer to like elements throughout the specification and drawings.

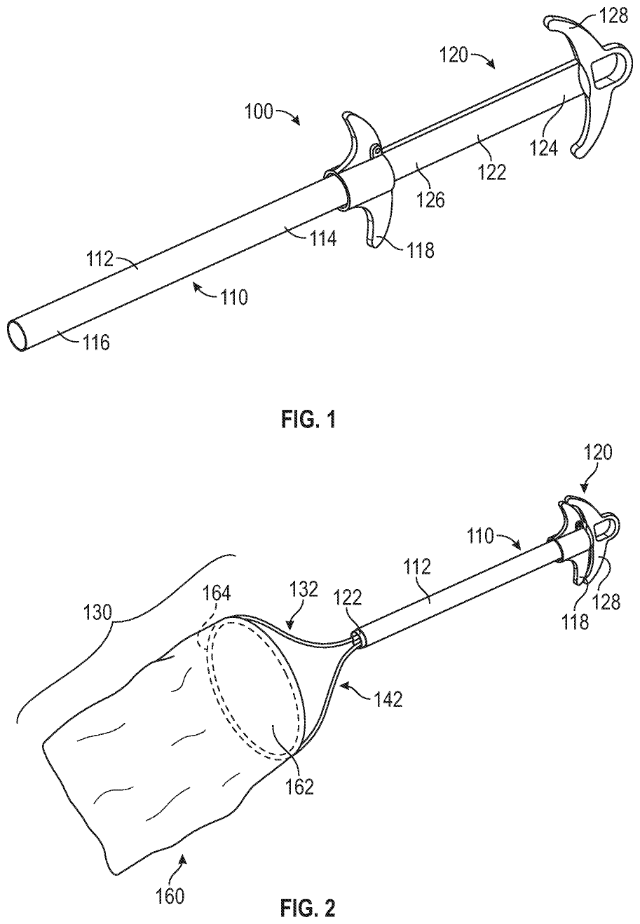

Referring to FIGS. 1-2, a tissue specimen retrieval device provided in accordance with the present disclosure is shown generally identified by reference numeral 100. Tissue specimen retrieval device 100 includes a first body 110, a second body 120, and an end effector assembly 130 including a specimen bag 160. First body 110 includes a first shaft 112 defining a proximal end portion 114 and a distal end portion 116. First body 110 further includes a first handle 118 disposed at proximal end portion 114 of first shaft 112. First handle 118 may be engaged with proximal end portion 114 of first shaft 112, monolithically formed with proximal end portion 114 of first shaft 112, or otherwise secured thereto in any suitable manner that enables a user to grasp and manipulate first handle 118 to thereby control manipulation of first shaft 112.

Second body 120 includes a second shaft 122 defining a proximal end portion 124 and a distal end portion 126. Second shaft 122 supports end effector assembly 130 at distal end portion 126 of second shaft 122 and is telescopically slidably within and relative to first shaft 112 between a retracted position of tissue specimen retrieval device 100 (FIG. 1), wherein end effector assembly 130 is disposed within first shaft 112, and a deployed position of tissue specimen retrieval device 100 (FIG. 2), wherein end effector assembly 130 extends distally from first shaft 112. Second body 120 further includes a second handle 128 disposed at proximal end portion 124 of second shaft 122. Second handle 128 may be engaged with proximal end portion 124 of second shaft 122, monolithically formed with proximal end portion 124 of second shaft 122, or otherwise secured thereto in any suitable manner that enables a user to grasp and manipulate second handle 128 to thereby control manipulation of second shaft 122. Second handle 128, more specifically, is movable relative to first handle 118 from a spaced-apart position (FIG. 1) to an approximated position (FIG. 2) to move tissue specimen retrieval device 100 from the retracted position (FIG. 1), wherein end effector assembly 130 is disposed within first shaft 112, to the deployed position (FIG. 2), wherein end effector assembly 130 extends distally from first shaft 112.

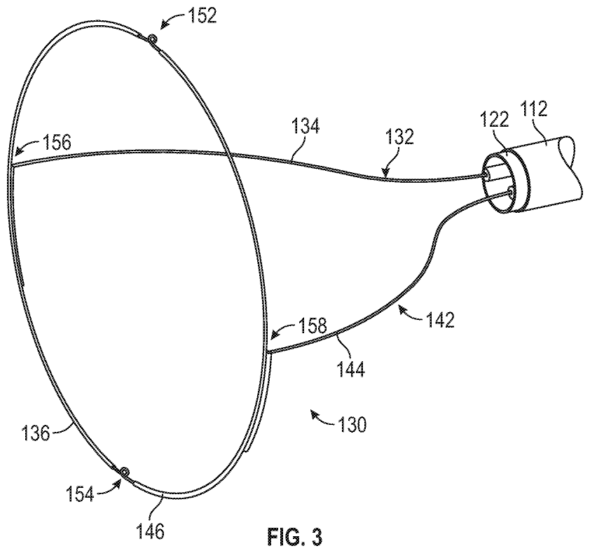

Referring to FIGS. 2 and 3, end effector assembly 130, as noted above, is supported at distal end portion 126 of second shaft 122. End effector assembly 130, more specifically, includes first and second arms 132, 142 extending distally from distal end portion 126 of second shaft 122 and a specimen bag 160 supported by and depending from first and second arms 132, 134. Each of first and second arms 132, 142 includes a proximal segment 134, 144 engaged with and extending distally from distal end portion 126 of second shaft 122, and a distal segment 136, 146 coupled to the respective proximal segment 134, 144 and extending distally therefrom.

Proximal segments 134, 144 of arms 132, 142, respectively, are substantially co-planar with one another so as to define a plane bisecting proximal segments 134 and 144, and are formed from resiliently flexible material, e.g., nitinol tubing, that biases proximal segments 134, 144 towards a first expanded position. Proximal segments 134, 144 each define, in the first expanded position, a curvature along at least a portion of the length thereof such that the proximal portions of proximal segments 134, 144 are relatively closer to one another and the distal portions of proximal segments 134, 144 are relatively father apart from one another.

Proximal segments 134, 144 of arms 132, 142, respectively, may be resiliently flexed from the first expanded position towards a first collapsed position, wherein the curvatures of proximal segments 134, 144 are at least partially eliminated and the distal portions of proximal segments 134, 144 are moved closer to one another, e.g., such that proximal segments 134, 144 are moved towards a substantially parallel orientation. Proximal segments 134, 144 are configured to resiliently flex within the plane defined thereby such that proximal segments 134, 144 remain substantially co-planar with one another in each of and during movement between the first expanded and first collapsed positions.

As an alternative or in addition to curved and/or resilient flexible configurations, proximal segments 134, 144 may define linear and/or substantially rigid configurations including one or more joints therealong to enable movement of proximal segments 134, 144 or portions thereof between the first expanded and first collapsed positions.

Distal segments 136, 146 of arms 132, 142, respectively, are substantially co-planar with one another so as to define a plane bisecting distal segments 136 and 146. Distal segments 136, 146 define curved configurations and are oriented relative to one another to cooperatively define a substantially oval-shaped configuration. Distal segments 136, 146 are formed from resiliently flexible material, e.g., nitinol tubing, that biases distal segments 136, 146 to a second expanded position, wherein distal segments 136, 146 cooperate to define a more-circular configuration.

Distal segments 136, 146 of arms 132, 142, respectively, are coupled to one another at first ends thereof via a first joint 152 and at second, opposite ends thereof via a second joint 154. In embodiments, first and second joints 152, 154 provide a bias that contributes to biasing distal segments 136, 146 towards the second expanded position; in other embodiments, the bias towards the second expanded position is provided by distal segments 136, 146 themselves without substantial bias imparted by first and second joints 152, 154. In either configuration, joints 152, 154 and distal segments 136, 146 are configured to move against the bias(es) such that distal segments 136, 146 are moved from the second expanded position, wherein distal segments 136, 146 cooperate to define the more-circular configuration, to a second collapsed position, wherein distal segments 136, 146 cooperate to define an elongated, oval-shaped configuration. Distal segments 136, 146 are configured to resiliently flex, and first and second joints 152, 154 are configured to move, within the plane defined by distal segments 136, 146, e.g., such that distal segments 136, 146 remain substantially co-planar with one another in each of and during movement between the second expanded and second collapsed positions. First and second joints 152, 154 may be hinge joints (living or multi-component hinges), pivot joints, torsion spring joints (similarly as detailed below), or other suitable joints.

As an alternative or in addition to distal segments 136, 146 cooperating to define an oval-shaped configuration and/or being resiliently flexible, distal segments 136, 146 may define linear and/or substantially rigid configurations including a plurality of joints, e.g., two to four joints, five joints, etc., defining a polygonal configuration, while still being movable between the second expanded position and the second collapsed position.

With reference to FIG. 2, distal segments 136, 146 support specimen bag 160 thereon with specimen bag 160 depending therefrom. Specimen bag 160 may be formed from any suitable bio-compatible material (or materials), e.g., ripstop nylon, configured to retain a tissue specimen therein. Specimen bag 160 defines at least one opening, e.g., open end 162 thereof, for receipt of a tissue specimen therein. Specimen bag 160 may include one or more channels 164 formed about at least a portion of the perimeter of open end 162 thereof for retaining distal segments 136, 146 of arms 132, 142, respectively, therein to support specimen bag 160 on distal segments 136, 146. Alternatively, open end 162 of specimen bag 160 may be welded, adhered, or otherwise affixed to or about distal segments 136, 146 to support specimen bag 160 thereon.

The one or more openings of specimen bag 160, e.g., open end 162, may include a cinch cord (not shown) disposed thereabout to enable selective closure of the opening. Specimen bag 160 may be disengaged from distal segments 136, 146 upon cinching closed open end 162 of specimen bag 160, retraction of end effector assembly 130 back towards the retracted position (FIG. 1), using a separate instrument, e.g., grasping device, and/or in any other suitable manner

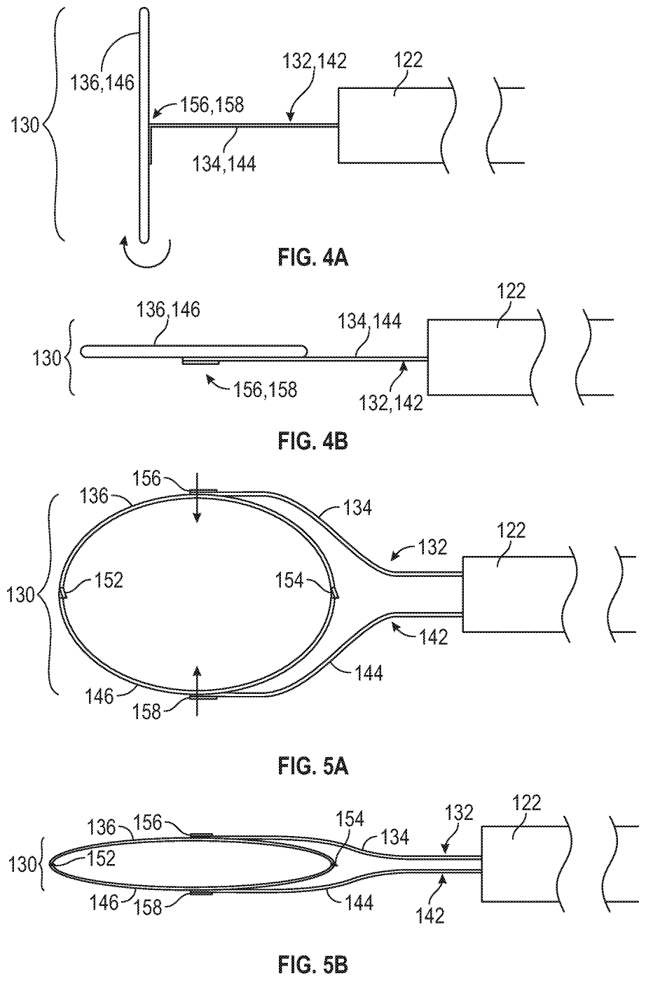

Turning again to FIGS. 2 and 3, distal segments 136, 146 of arms 132, 142 are coupled to respective proximal segments 134, 144 of arms 132, 142 via third and fourth joints 156, 158, respectively, to enable the collapsing of end effector assembly 130 wherein distal segments 136, 146 are collapsed onto to proximal segments 134, 144. Third and fourth joints 156, 158 may be hinge joints (living or multi-component hinges), pivot joints, torsion spring joints (similarly as detailed below), or other suitable joints. Third and fourth joints 156, 158, more specifically, enable collapsing of distal segments 136, 146 relative to proximal segments 134, 144 from a third expanded position (see FIG. 4A), wherein the planes defined by distal segments 136, 146 and proximal segments 134, 144 are disposed in a first orientation relative to one another, to a third collapsed position (see FIG. 4B), wherein the planes defined by distal segments are disposed in a second orientation relative to one another. Third and fourth joints 156, 158 are configured to bias distal segments 136, 146 towards the third expanded position relative to proximal segments 134, 144, wherein the planes defined thereby are disposed in the first orientation, although other configurations are also contemplated.

In embodiments, in the third expanded position, corresponding to the first orientation of the planes defined by distal segments 136, 146 and proximal segments 134, 144, the planes are disposed in substantially perpendicular orientation relative to one another; in other embodiments, the planes define an angle therebetween of approximately 45 degrees to approximately 120 degrees; in still other embodiments, the planes define an angle therebetween of approximately 60 degrees to approximately 105 degrees; and in yet other embodiments, the planes define an angle therebetween of approximately 75 degrees to approximately 90 degrees.

In embodiments, in the third collapsed position, corresponding to the second orientation of the planes defined by distal segments 136, 146 and proximal segments 134, 144, the planes are disposed in substantially parallel orientation relative to one another; in other embodiments, the planes define an angle therebetween of approximately 0 degrees to approximately 15 degrees; in still other embodiments, the planes define an angle therebetween of approximately 0 degrees to approximately 10 degrees; and in yet other embodiments, the planes define an angle therebetween of approximately 0 degrees to approximately 5 degrees.

Turning back to FIGS. 1 and 2, in the retracted position of tissue specimen retrieval device 100 (FIG. 1), as noted above, end effector assembly 130 is disposed within first shaft 112 of first body 110. In order to fit end effector assembly 130 within first shaft 112 in the retracted position of tissue specimen retrieval device 100 (FIG. 1), the end effector assembly 130 is collapsed from two orientations. Although described herein in terms of a first collapse followed by a second collapse, the present disclosure also contemplates that the first collapse follows the second collapses, or that at least portions of the first and second collapses are effected substantially simultaneously.

Referring to FIGS. 4A and 4B, the first collapse corresponds to the movement of distal segments 136, 146 of arms 132, 142 about third and fourth joints 156, 158, respectively, and relative to respective proximal segments 134, 144 from the third expanded position to the third collapsed position, as detailed above. This movement of distal segments 136, 146 about third and fourth joints 156, 158 to effect the first collapse occurs via relative movement of the planes defined by proximal segments 134, 144 and distal segments 136, 146, e.g., the plane defined by distal segments 136, 146 is moved relative to the plane defined by proximal segments 134, 144 during the first collapse.

The second collapse, illustrated in FIGS. 5A and 5B, is a compound collapse corresponding to the flexion and movement about joints 152, 152 of distal segments 136, 146 of arms 132, 142, respectively, from the second expanded position to the second collapsed position, as detailed above, as well as flexion of proximal segments 134, 144 of arms 132, 142 from the first expanded position to the first collapsed position, as also detailed above. These movements corresponding to the second collapse occur within the planes defined by proximal segments 134, 144 and distal segments 136, 146, respectively such that the planes defined by proximal segments 134, 144 and distal segments 136, 146 remain substantially stationary relative to one another during the second collapse.

With additional reference to FIGS. 1 and 2, once the first and second collapses are effected, as detailed above, specimen bag 160 may be folded, twisted, wrapped, rolled, and/or otherwise manipulated relative to arms 132, 142 and, thereafter, second shaft 122 may be pulled proximally relative to first shaft 112, e.g., via moving second handle 128 away from first handle 118, thereby drawing end effector 130 into first shaft 112 to the retracted position. As an alternative to initially manipulating specimen bag 160 relative to arms 132, 142 before drawing end effector 130 into first shaft 112, arms 132, 142 of end effector assembly 130 may instead be at least partially drawn into first shaft 112 followed by manipulating specimen bag 160 to fit within first shaft 112. In other embodiments, rather than effecting the first and second collapses in the deployed position and the retracting end effector assembly 130 proximally into first shaft 112, end effector assembly 130, after the first and second collapses are effected, may be inserted through the proximal end portion 114 of first shaft 112 and moved therethrough to the retracted position, e.g., via manipulating second handle 128. Other suitable configurations for loading end effector assembly 130 within first shaft 112 are also contemplated. Regardless of the loading configuration, once loaded within first shaft 112 in the retracted position, the first and second collapses of arms 132, 142 of end effector assembly 130 are maintained against the biases thereof via the internal spatial constraints of first shaft 112.

Continuing with reference to FIGS. 1 and 2, with end effector assembly 130 loaded within first shaft 112 and tissue specimen retrieval device 100 disposed in the retracted position (FIG. 1), tissue specimen retrieval device 100 is ready for use. More specifically, tissue specimen retrieval device 100 may be inserted into an internal surgical site, e.g., through a suitable access device (not shown), and thereafter moved from the retracted position to the deployed position, e.g., via grasping second handle 128 and moving second handle 128 towards first handle 118.

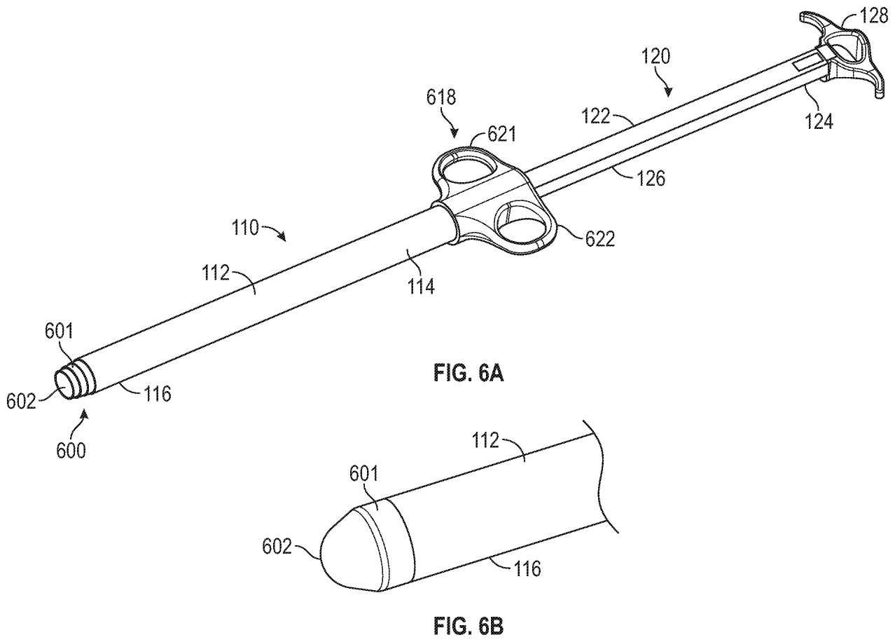

As end effector assembly 130 is deployed from first shaft 112 and, thus, is no longer constrained by first shaft 112, proximal segments 134, 144 of arms 132, 142 are returned from the first collapsed position to the first expanded position, distal segments 136, 146 of arms 132, 142 are returned from the second collapsed position back to the second expanded position, and distal segments 136, 146 are returned relative to proximal segments 134, 144 from the third collapsed position to the third expanded position. The return to the first, second, and third expanded positions may occur in any order and/or portions thereof may occur substantially simultaneously. Further, upon return to the first, second, and/or third expanded positions, specimen bag 160 is unfurled and open end 162 thereof presented to facilitate insertion of a tissue specimen therein during use. Unless specified to the contrary below, the tissue specimen retrieval device described in more detail below with reference to FIGS. 6A to 8B is substantially the same as the specimen retrieval device 100 described above. Thus, wherever technically feasible, the features described with respect to the specimen retrieval device 100 are similarly available to the tissue specimen retrieval device described in more detail below with reference to FIGS. 6A to 8B.

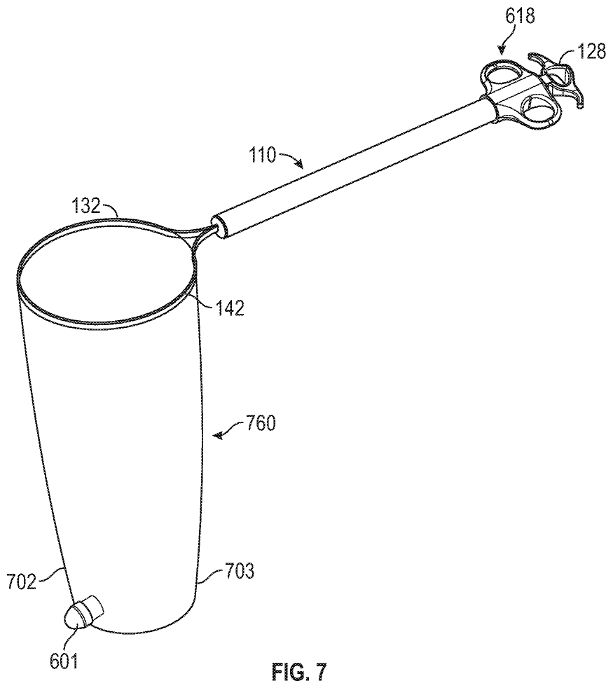

Referring to FIGS. 6A, 6B, 7, 8A and 8B, a tissue specimen retrieval device includes a first shaft 112 and a second shaft 122 telescopically movable relative to the first shaft 112. The second shaft 122 supports an end effector assembly 600 at a distal end portion 116 thereof. The second shaft 122 is movable relative to the first shaft 112 between a retracted position (see, e.g., FIG. 6A), in which the end effector assembly 600 is disposed within the first shaft 112, and a deployed position (see, e.g., FIG. 7), in which the end effector assembly 600 extends distally from the first shaft 112. Movement of the second shaft 122 from the retracted position to the deployed position advances the end effector assembly 600 out of the first shaft 112 and deploys the end effector assembly 600 including a tissue specimen bag 760, which is described in more detail below. Thus, the tissue specimen bag 760 is positioned along an interior space of the first shaft 112 when the second shaft 122 is in the retracted position. The tissue specimen bag 760 is in a furled configuration while positioned in the first shaft 112 and unfurls when deployed from the first shaft 112.

The end effector assembly 600 includes the tissue specimen bag 760 supported by a first arm 132 and a second arm 142. The first and second arms 132 and 142 can be deployed to open the tissue specimen bag 760 when the second shaft 122 is in the deployed position. The tissue specimen bag 760 includes an insertion cap 601 disposed on and extending outwardly from an exterior surface 702 of the tissue specimen bag 760 (see, e.g., FIG. 7). The insertion cap 601 extends from the distal portion 116 of the first shaft 112 when the second shaft 122 is in the retracted position to define an atraumatic insertion tip 602 (see, e.g., FIGS. 6A and 6B). The insertion cap 601 may be employed, for example, to insert the tissue specimen bag 760 abdominally during a laparoscopic surgery (e.g., via an insertion hole formed by a trocar), or vaginally.

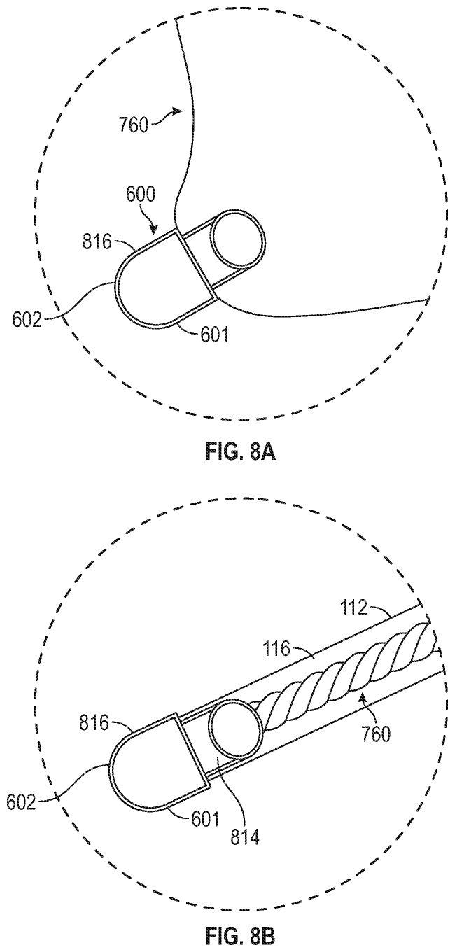

In some aspects, the insertion cap 601 includes a proximal portion 814 that can be inserted into the distal portion 116 of the first shaft 112 when the second shaft 122 is in the retracted position. The insertion cap 601 also includes a rounded distal portion 816 (see, e.g., FIGS. 8A and 8B).

In some aspects, an outer circumference of the rounded distal portion 816 of the insertion cap 601 is substantially aligned with an outer circumference of the distal portion 116 of the first shaft 112 when the second shaft 122 is in the retracted position (see, e.g., FIG. 6B). Thus, the rounded distal portion 816 may be aligned with an outer surface of the first shaft 112 to create a continuous smooth surface for atraumatic insertion.

In some aspects, the insertion cap 601 is welded to the tissue specimen bag 760. For example, the insertion cap 601 may be welded to a sidewall of the tissue specimen bag 760.

In some aspects, the insertion cap 601 is positioned at a bottom portion 703 of the tissue specimen bag 760 (see, e.g., FIG. 7). The insertion cap 601 protrudes from the exterior surface 702 of the tissue specimen bag 760 at the bottom portion 703 of the tissue specimen bag 760.

In some aspects, a first handle 618 is disposed at a proximal portion 114 of the first shaft 112 and a second handle 128 is disposed at a proximal portion 124 of the second handle 122. The first and second handles 618 and 128 are relatively movable to move the second shaft 122 between the retracted and deployed positions. The first handle 618 includes first and second finger loops 621 and 622. The finger loops 621 and 622 allow a user to hold the first shaft 112 in position while the second shaft 122 is telescopically advanced to move the second shaft 122 between the retracted and deployed positions.

In some aspects, the proximal portion 814 of the insertion cap 601 has a narrower width than a width of the rounded distal portion 816 of the insertion cap 601 (see, e.g., FIGS. 8A and 8B). A distal surface of the insertion cap 601 may be in direct contact with a distalmost end of the first shaft 112 when the second shaft 122 is in the retracted position.

In some aspects, the insertion cap 601 (e.g., including the atraumatic insertion tip 602) includes or is formed of plastic or silicon. However, exemplary embodiments of the present disclosure are not limited thereto and other materials may be employed to form the insertion cap 601.

In some aspects, the first and second arms 132 and 142 of the end effector assembly 600 are configured to close the tissue specimen bag 760 when the second shaft 122 is moved to the retracted position.

In some aspects, the first and second arms 132 and 142 of the end effector assembly 600 are configured to deploy the insertion cap 601 and the tissue specimen bag 760 from the first shaft 112 when the second shaft 122 is moved to the deployed position.

The insertion cap 601 having a substantially round end shape and defining the atraumatic tip 602 may be formed of plastic or silicon.

According to aspects of the present disclosure, the insertion cap 601 allows the end or bottom of the tissue specimen bag 760 to be locatable because the insertion cap 601 is the first part to enter an insertion hole upon deployment and the insertion cap 601 is reinserted into the distal end 116 of the first shaft 112 when the tissue specimen bag 760 is furled and is reinserted into the first shaft 112.

In some aspects, the insertion cap 601 is locatable. The insertion cap 601 may be used to determine a location of the bottom portion 703 of the tissue specimen bag 760. The insertion cap 601 may be locatable by visual inspection using a laparoscopic camera, or by an electromagnetic tracking system.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A tissue specimen retrieval device, comprising:
a first shaft;
a second shaft telescopically movable relative to the first shaft, the second shaft supporting an end effector assembly at a distal end portion thereof and movable relative to the first shaft between a retracted position, wherein the end effector assembly is disposed within the first shaft, and a deployed position, wherein the end effector assembly extends distally from the first shaft,
the end effector assembly including a tissue specimen bag supported by a first arm and a second arm, the first and second arms configured to open the tissue specimen bag when the second shaft is in the deployed position, the tissue specimen bag including:
an insertion cap disposed on and extending outwardly from an exterior surface of the tissue specimen bag, the insertion cap configured to operably engage the distal portion of the first shaft when the second shaft is in the retracted position to define an atraumatic insertion tip.

2. The tissue specimen retrieval device of claim 1, wherein the insertion cap includes:
a proximal portion configured to be inserted into a distal portion of the first shaft when the second shaft is in the retracted position; and
a rounded distal portion.

3. The tissue specimen retrieval device of claim 2, wherein an outer circumference of the rounded distal portion of the insertion cap is configured to substantially align with an outer circumference of the distal portion of the first shaft when the second shaft is in the retracted position.

4. The tissue specimen retrieval device of claim 1, wherein the insertion cap is operably engaged with the tissue specimen bag.

5. The tissue specimen retrieval device of claim 1, wherein the insertion cap is positioned at a bottom portion of the tissue specimen bag.

6. The tissue specimen retrieval device of claim 1, wherein the tissue specimen bag is disposed within the first shaft when the second shaft is in the retracted position.

7. The tissue specimen retrieval device of claim 1, further comprising a first handle disposed at a proximal portion of the first shaft and a second handle disposed at a proximal portion of the second shaft, wherein the first and second handles are relatively movable to move the second shaft between the retracted and deployed positions.

8. The tissue specimen retrieval device of claim 7, wherein the first handle includes first and second finger loops.

9. The tissue specimen retrieval device of claim 2, wherein the proximal portion of the insertion cap has a narrower width than a width of the rounded distal portion of the insertion cap.

10. The tissue specimen retrieval device of claim 1, wherein the insertion cap is locatable to facilitate location of a bottom portion of the tissue specimen bag.

11. The tissue specimen retrieval device of claim 1, wherein the insertion cap includes plastic or silicon.

12. The tissue specimen retrieval device of claim 2, wherein the rounded distal portion of the insertion cap is formed of plastic or silicon.

13. The tissue specimen retrieval device of claim 1, wherein the first and second arms of the end effector assembly are configured to close the tissue specimen bag when the second shaft is in the retracted position.

14. The tissue specimen retrieval device of claim 1, wherein the first and second arms of the end effector assembly are configured to deploy the insertion cap and the tissue specimen bag from the first shaft when the second shaft is in the deployed position.

15. A tissue specimen retrieval device, comprising:
a first shaft;
a second shaft telescopically movable relative to the first shaft, the second shaft supporting an end effector assembly at a distal end portion thereof and movable relative to the first shaft between a retracted position, wherein the end effector assembly is disposed within the first shaft, and a deployed position, wherein the end effector assembly extends distally from the first shaft,
the end effector assembly including a tissue specimen bag supported by a first arm and a second arm, the first arm including a first hinge and the second arm including a second hinge, the first and second arms configured to open the tissue specimen bag about the first and second hinges, respectively, when the second shaft is in the deployed position, the tissue specimen bag including:
an insertion cap disposed on and extending outwardly from an exterior surface of the tissue specimen bag, the insertion cap configured to operably engage the distal portion of the first shaft when the second shaft is in the retracted position to define an atraumatic insertion tip.

16. The tissue specimen retrieval device of claim 15, wherein the insertion cap includes:
a proximal portion configured to be inserted into a distal portion of the first shaft when the second shaft is in the retracted position; and
a rounded distal portion.

17. The tissue specimen retrieval device of claim 16, wherein an outer circumference of the rounded distal portion of the insertion cap is configured to substantially align with an outer circumference of the distal portion of the first shaft when the second shaft is in the retracted position.

18. The tissue specimen retrieval device of claim 15, wherein the insertion cap is operably engaged with the tissue specimen bag.

19. The tissue specimen retrieval device of claim 15, wherein the insertion cap is positioned at a bottom portion of the tissue specimen bag.

20. The tissue specimen retrieval device of claim 15, wherein the tissue specimen bag is disposed within the first shaft when the second shaft is in the retracted position.
